(19) United States
(12) Patent Application Publication
(10) Pub. No.: US 2013/0190591 A1
(43) Pub. Date: Jul. 25, 2013

(54) PHOTOACOUSTIC TRANSDUCER AND IMAGING SYSTEM

(71) Applicants: Desmond Hirson, Thornhill (CA); Andrew Needles, Toronto (CA); Pinhas Ephrat, St. Catharines (CA); Nicholas Christopher Chaggares, Whitby (CA)

(72) Inventors: Desmond Hirson, Thornhill (CA); Andrew Needles, Toronto (CA); Pinhas Ephrat, St. Catharines (CA); Nicholas Christopher Chaggares, Whitby (CA)

(21) Appl. No.: 13/695,275

(22) PCT Filed: Apr. 29, 2011
(86) PCT No.: PCT/US11/34640

§ 371 Date: Apr. 9, 2013

Related U.S. Application Data
(60) Provisional application No. 61/329,979, filed on Apr. 30, 2010.

(51) Int. Cl. A61B 5/00

(52) U.S. Cl. CPC A61B 5/0095; A61B 5/742
USPC 600/407

(57) ABSTRACT

The invention disclosed herein features a photoacoustic scan head that includes laser fibers integrated into the housing of an arrayed ultrasound transducer using an optically transparent epoxy or other resin. The light-emitting ends of the fibers are positioned adjacent to the front surface of the transducer and direct laser light onto a subject being scanned by the transducer. The light beams generated by the fibers may be angled to intersect the acoustic field generated by the transducer so as to generate a photoacoustic effect in the region scanned by the transducer.

### FIELD OF THE INVENTION

The present invention generally relates to the fields of photoacoustic imaging and medical diagnostics. More specifically, the present invention relates to a photoacoustic imaging system that includes an ultrasound transducer with integrated optical fibers that can be used to obtain photoacoustic images of a subject, such as a human or small laboratory animal, for diagnostic and other medical or research purposes.

### BACKGROUND

Ultrasound-based imaging is a common diagnostic tool used by medical professionals in various clinical settings to visualize a patient's muscles, tendons and internal organs, as well as any pathological lesions that may be present, with real time tomographic images. Ultrasonic imaging is also used by scientists and medical researchers conducting in vivo studies to assess disease progression and regression in test subjects.

Ultrasound imaging systems typically have a transducer that sends and receives high frequency sound waves. The transducer often utilizes a piezoelectric component that is able to convert received ultrasound waves into an electrical signal. A central processing unit powers and controls the system components, processes signals received from the transducer to generate images, and displays the images on a monitor.

Ultrasound imaging is relatively quick, portable and inexpensive compared to other types of imaging modalities, such as MRI. It is also less invasive with fewer potential side effects than modalities using ionizing radiation, such as x-Ray and PET. However, conventional ultrasound technology has limitations that make it unsuitable for some applications. For example, ultrasound waves do not pass well through certain types of tissues and anatomical features, and ultrasound images typically have poorer contrast than X-Ray and MRI images. Also, ultrasonic imaging has difficulties distinguishing between acoustically homogenous tissues (i.e. tissues having similar ultrasonic properties).

Photoacoustic imaging is a modified form of ultrasound imaging that is based on the photoacoustic effect, in which the absorption of electromagnetic energy, such as light or radio-frequency waves, generates acoustic waves. In photoacoustic imaging, laser pulses are delivered into biological tissues (when radio frequency pulses are used, the technology is usually referred to as thermoacoustic imaging). A portion of the delivered energy is absorbed by the tissues of the subject and converted into heat. This results in transient thermoelastic expansion and thus wideband (e.g. -MHz) ultrasonic emission. The generated ultrasonic waves are then detected by ultrasonic transducers to form images. Photoacoustic imaging has the potential to overcome some of the problems of pure ultrasound imaging by providing, for example, enhanced contrast and improved specificity. At the same time, since non-ionizing radiation is used to generate the ultrasonic signals, it has fewer potentially harmful side effects.

Different techniques have been used for shining laser light adjacent to an ultrasound transducer to initiate the photoacoustic effect. In reflection mode photoacoustics, where the light is directed to the tissue from the same side as the transducer, the most common approach is similar to those used in dark field microscopy and takes the form of optical lenses and mirrors to focus the light in a concentric circle around the transducer. Although well suited for a single round transducer, this approach is less suitable for a rectangular linear array of transducers, because the light distribution becomes uneven in the array's field of view. Another challenge associated with prior methods of photoacoustic imaging is that of laser pulse-to-pulse intensity variation. Pulse-to-pulse variation results in undesired fluctuations in acoustic intensity across a photoacoustic image and between successive images. Unless it is quantified and normalized, such pulse-to-pulse variation can have an adverse effect on the quality and reliability of the photoacoustic images.

In view of the limitations of current photoacoustic imaging methods, there remains a need for photoacoustic systems and techniques that provide an easy and convenient approach for providing laser light to a subject for obtaining photoacoustic images.

### SUMMARY OF THE INVENTION

The present invention features a photoacoustic scan head for obtaining photoacoustic images of a target. The scan head comprises a transducer housing that contains an arrayed ultrasound transducer that transmits and/or receives ultrasound waves to and/or from the target. The scan head also includes a plurality of optical fibers for directing laser light to the target. The light-emitting ends of the fibers are positioned adjacent to the front surface of the transducer and integrated into the nosepiece of the housing with an optically transparent resin.

Typically, the optical fibers in the housing are joined together to form a bundle or cable. This bundle or cable may further include one or more electrical wires to form a coaxial cable. The electrical wires of the coaxial cable run from the transducer located in the nosepiece of the scan head to a connector that interfaces with an ultrasound transceiver or beamformer. The optical fibers run from one or more positions adjacent to the transducer to a connector that interfaces with a laser system.

In certain implementations of the invention, the light-emitting end of the bundle of fibers may be divided into two or more groups of fibers that are positioned next to the transducer within the nosepiece of the housing. For example, the optical fibers may be arranged into two separate bundles with the light-emitting end of each bundle in the form of a rectangular bar of fibers. Each bar of fibers may be symmetrically positioned along opposite sides of the ultrasound transducer. Alternatively, the light-emitting ends of each bundle may take the form of a circle or other suitable shape for providing a beam of light.

Other arrangements of the optical fibers in the scan head are also possible. For example, the optical fibers may be separated into more than two bundles, and/or may be arranged symmetrically or asymmetrically alongside each of the edges of the front surface of the transducer. The fibers may be positioned along an entire edge or only a portion of an edge of the front surface of the transducer. In addition, optical fibers can be arranged around the transducer in any of a variety of shapes or configurations, such as rectangles, squares, circles, etc.

The light-emitting end of each bundle of optical fibers may be positioned at any desired angle relative to the front surface of the arrayed ultrasound transducer. Typically the bundles of optical fibers are positioned such that the beam of light generated by each bundle intersects a plane that runs perpendicular to the front face of the transducer. In some embodiments, multiple elevation angles may be used.

Typically, the ultrasound transducer in the scan head is an arrayed transducer that has a plurality of transducer elements for generating and receiving ultrasound waves. Suitable arrayed transducers include, for example, linear array transducers, phased array transducers, two-dimensional array transducers, and curved array transducers. Other types of fixed transducers may also be used

In some embodiments of the invention, the ultrasound transducer is a high frequency transducer that receives and/or transmits ultrasound at a frequency from about 15 MHz to about 100 Mhz. Most typically, the transducer receives and/or transmits ultrasound at a frequency of at least 20 MHz.

The photoacoustic scan head of the invention may optionally further include a real-time capable photo-sensor for monitoring pulse-to-pulse laser energy, such as reflected or backscattered energy from the subject. The photo-sensor can be integrated into the nosepiece of the housing using the same optically transparent resin used to integrate the optical fibers into the housing. In addition, a separate group of optical fibers may be positioned next to the photo-sensor so as to emit a beam of light onto an area of the target adjacent to the acoustic field generated by the ultrasound transducer. Also, a plurality of photo-sensors may be distributed inside the nosepiece for monitoring of pulse-to-pulse energy variation at different regions of the arrayed ultrasound transducer. Alternatively, the photo-sensor may be separate from the scan head and located outside the transducer housing.

The optical fibers are preferably integrated into the nosepiece of the scan head using an optically transparent resin. The resin is typically an epoxy or other polymer resin. In some implementations of the invention, it is desirable to use a resin that has an index of refraction that matches that of the optical fibers. The resin may also be used to integrate other components of the device into the nosepiece, including the ultrasound transducer and optional photo-sensor.

In one embodiment of the invention, the translucent resin used to integrate the optical fibers into the scan head also acts as a lens to focus the beams of light emitted by the optical fibers. Such lenses can be used to provide beams of light with a depth of focus that matches that of the acoustic field generated by the arrayed ultrasound transducer.

In another aspect, the invention features a photoacoustic imaging system that comprises (i) a photoacoustic scan head as described above that includes an arrayed ultrasound transducer with integrated bundle(s) of optical fibers; (ii) a laser system connected to the optical fibers for generating pulses of non-ionizing light; (iii) an ultrasonic transceiver or beamformer connected to the transducer of the scan head, (iv) a computer for controlling system components and processing received ultrasound data into an image, and (iv) a monitor for displaying the image.

The photoacoustic imaging system of the invention may be used to image various organs (e.g., heart, kidney, brain, liver, blood, etc.) and/or tissue of a subject, or to image a neo-plastic condition or other disease condition of the subject. Typically the subject is a mammal, such as a human. The invention is also particularly well-suited for imaging small animals, such as laboratory mice and/or rats.

The above summary is not intended to describe each embodiment or every implementation of the invention. Other embodiments, features, and advantages of the present invention will be apparent from the following detailed description thereof, from the drawings, and from the claims. It is to be understood that both the foregoing summary and the following detailed description are exemplary and explanatory only and are not restrictive of the invention as claimed.

### BRIEF DESCRIPTION OF THE DRAWINGS

The invention may be more completely understood in consideration of the accompanying drawings, which are incorporated in and constitute a part of this specification, and together with the description, serve to illustrate several embodiments of the invention:

FIG. 1 is a side view of a fiber optic bundle that is bifurcated at one end for use in a photoacoustic scan head;

FIGS. 2a and 2b are perspective views of a photoacoustic scan head with an integrated fiber optic cable;

FIG. 3a is a side view and FIG. 3b is a front view of a photoacoustic scan head having a fixed transducer and integrated bundles of optical fibers;

FIGS. 4a and 4b are side views of the nosepiece of a photoacoustic scan head showing the optical and acoustic fields generated by the scan head;

FIG. 5 is a cross-sectional side view of the nosepiece of a photoacoustic scan head showing the acoustic field generated by the transducer and the light beams generated by the optical fibers;

FIGS. 6a, 6b, and 6c are side views (b and c show cross-sections) of the scan head depicting the acoustic field generated by the transducer and the light beams generated by the optical fibers;

FIGS. 6d, 6e, and 7f are top views (e and f show cross-sections) of the scan head depicting the acoustic field generated by the transducer and the light beams generated by the optical fibers; and

FIG. 7 is a block diagram showing an embodiment of a photoacoustic imaging system that includes a scan head attached to an ultrasound transceiver and a laser system.

While the invention is amenable to various modifications and alternative forms, specifics thereof have been shown by way of example in the drawings. It should be understood, however, that the intention is not to limit the invention to the particular embodiments depicted in the drawings or in the accompanying description. On the contrary, the intention is to cover all modifications, equivalents, and alternatives falling within the spirit and scope of the invention.

### DETAILED DESCRIPTION

The present invention provides a photoacoustic scan head that includes laser fibers integrated into the housing of an arrayed ultrasonic transducer to allow for the delivery of uniform light energy to an acoustic imaging plane generated by the transducer. In particular, the laser fibers, which may be arranged, for example, in rectangular shaped bundles, are embedded into the housing of the transducer alongside the ultrasound elements. The integrated fiber bundle(s) are potted into the housing using a transparent potting epoxy or other resin selected to provide sufficient refraction for lens effects to be used to allow precise illumination uniformly along the acoustic imaging plane. In addition, multiple angles of illumination can be incorporated by shaping the face of the epoxy or other resin material used to pot the bundled fibers in the transducer housing. This allows the light to be delivered at a specific angle relative to the face of the transducer.

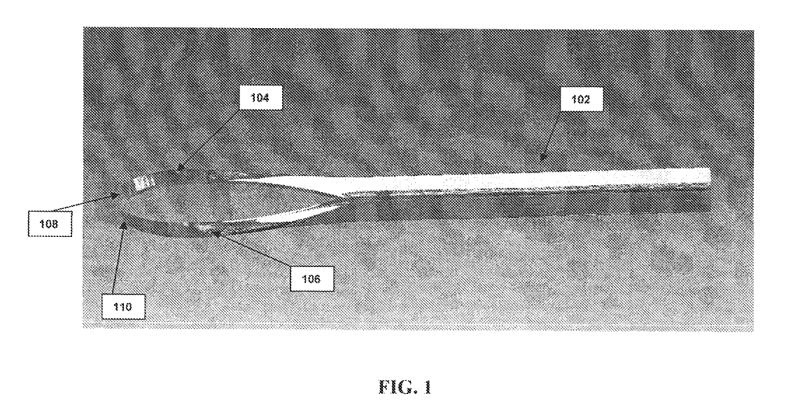

An example of a laser fiber bundle that may be integrated into an ultrasound transducer housing in accordance with the invention is shown in FIG. 1. The laser bundle 102 is made up of a plurality of optical fibers that have been joined together to form a cable that runs from the scan head to a connector that interfaces with a laser system. The end of the bundle 102 is bifurcated into separate bundles 104 and 106, which form two light-emitting ends 108 and 110. The bundles 104 and 106 are randomized for uniform light distribution and the light-emitting ends 108 and 110 are arranged into rectangular bars that can be integrated, e.g. with an epoxy or other resin material, into the transducer housing.

In one embodiment of the invention, the light-emitting bars 108 and 110 are arranged symmetrically in relation to the front of the transducer. In particular, a single rectangular light bar is placed on each side of the transducer array elements so that they produce beams that cross in front of the ultrasound transducer thus forming a plane of intersection perpendicular to the face of the transducer. The optical fibers can be potted into the nosepiece of the transducer having been first set into a mold designed to create a smoother face on the nose of the composite transducer and to create an interior pocket that will be used to align the acoustic array. The potting may be done using a transparent epoxy or other resin such that lenses may be formed in front of the light bars using the mold to shape the epoxy or other resin material. The ultrasonic array is then aligned and potted into the pocket previously formed when potting the fibers. This allows the light bars to be positioned symmetrically on either side of the acoustic transducer, and in close proximity to each other, so that the beams of the light bars can cross along a plane perpendicular to the acoustic transducer and contained in the imaging plane of the ultrasonic array from as shallow a depth as possible, thereby maximizing the volume over which the photoacoustic imaging can take place. The depth of the region over which the optical beams cross and the angle at which they converge can be arranged to optimize the photoacoustic effect.

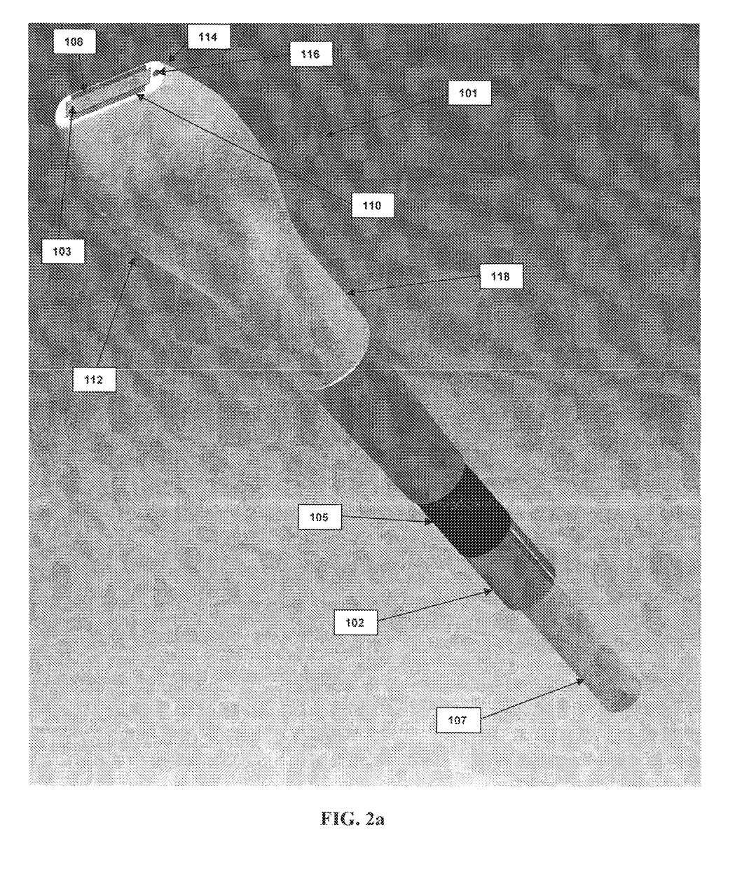

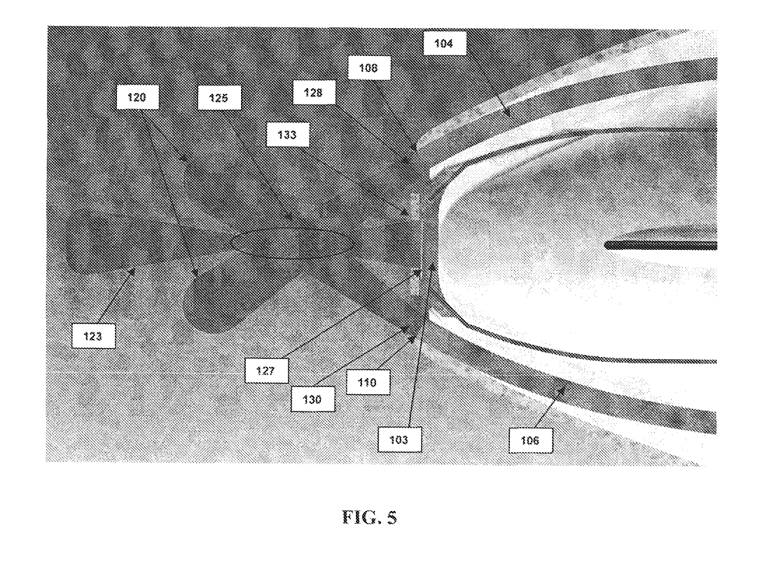

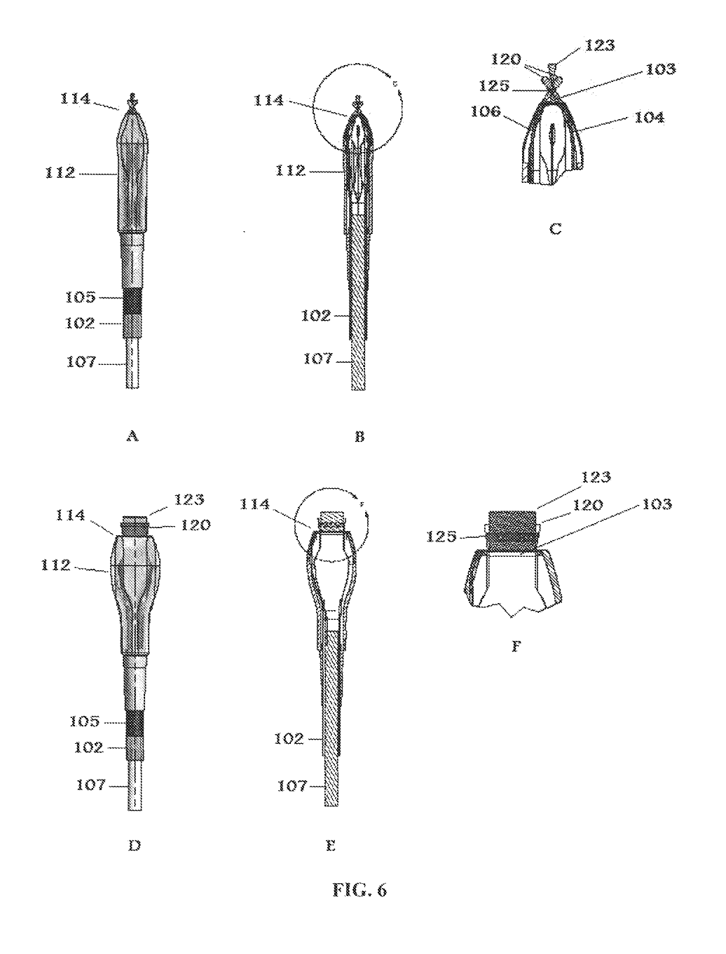

FIGS. 2-6 show an embodiment of a photoacoustic scan head 101 constructed in the above-described manner. The nosepiece 114 of the scan head 101 has an arrayed ultrasound transducer 103 for transmitting and receiving ultrasonic waves. The scan head 101 also includes a fiber optic cable 105 that includes a plurality of optical fibers 102. At one end, the bundle of optical fibers 102 is bifurcated into two groups of fibers that are formed into light-emitting bars 108 and 110 that are positioned on opposite sides of the arrayed transducer 103. The bars 108 and 110 direct laser light onto the target to generate ultrasonic waves which are detected by the arrayed transducer 103. Although shown in the figures as rectangular bars, these groups of fibers may be formed into any other suitable shape, such as circle, oval, square, triangle, etc., to produce beams of light. The laser light emitted from the optical fibers travels to an illumination region on the skin surface of the subject to be imaged, and generates ultrasonic waves within the tissue of the subject.

The various components of the scan head 101 are encased with a protective housing 112. The housing may be made of a plastic or other suitable rigid or semi-rigid material, and may be shaped to provide for hand held use.

As shown in FIG. 2, the electrical wires 107 supplying the ultrasonic array can be arranged in the center of the fiber optic bundle 102 such that a composite wire/fiber optic coaxial cable 105 is formed. The rear housing 118 is then fitted over the nosepiece 114 and cables/connectors with a strain relief so that the user experiences a transducer having a single cable 105 exiting the scan head 101. At the far end the cable finishes with an optical connector and an electronic connector that interface with a laser-generating system and ultrasonic transceiver/beamformer respectively.

The nosepiece 114 of the scan head 101 may also include a photo-sensor 116, such as an integrated photo-diode based monitoring device to capture, for example, the backscattered light from the surface of the skin. By integrating the monitoring system in the nosepiece 114 of the scan head 101, photoacoustic data can be normalized so that pulse-to-pulse laser intensity variation is mitigated in real-time. The photo-sensor may also be potted into a location at one or both ends of the acoustic lens using an optically transparent epoxy or polymer resin, and may be recessed and/or angled so that it can measure the illumination of the tissue immediately at the end of the array.

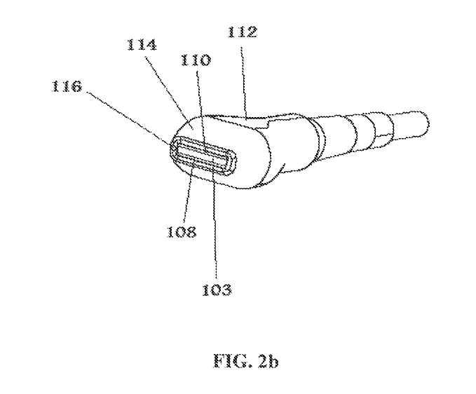

FIGS. 2 and 3 show a single photo-sensor 116 potted at one end of the arrayed transducer 103, and aimed to look at the imaged subject. The light bars 108 and 110 can be arranged so that they extend slightly beyond the end of the acoustic lens to give the photo-sensor greater illumination, if required, and to ensure that light conditions at the surface of the tissue correspond closely to the light conditions of the tissue under the acoustic lens. Furthermore, the fiber optic cable may be further split into, e.g., two smaller fiber bundles that shine light onto an area adjacent to the acoustic transducer, with the photo-sensor positioned in between the bundles so that it can measure an optical field under the same geometric conditions as the photoacoustic effect takes place.

In an alternative embodiment, the photo-sensor may be separate from the scan head (i.e. located outside the transducer housing). For example, the photosensor may be located as part of the cart assembly for the laser system that supplies laser light for the optical fibers. By using the optical fiber bundles to guide backscattered light back to a photo-sensor that is situated outside of the transducer housing it may be possible to achieve a more uniform sampling of backscattered light, and to use a larger photo-sensor than would be able to fit inside the transducer housing.

In yet another alternative embodiment, the photo-sensor may again be separate from the scan head with the photo-sensor being located as part of the cart assembly for the laser system. However, rather than using the existing optical fiber bundles to guide backscattered light back to a photo-sensor situated outside the transducer housing, additional fibers dedicated solely to directing light back to the photo-sensor could be placed either within the existing light bars or around their exterior.

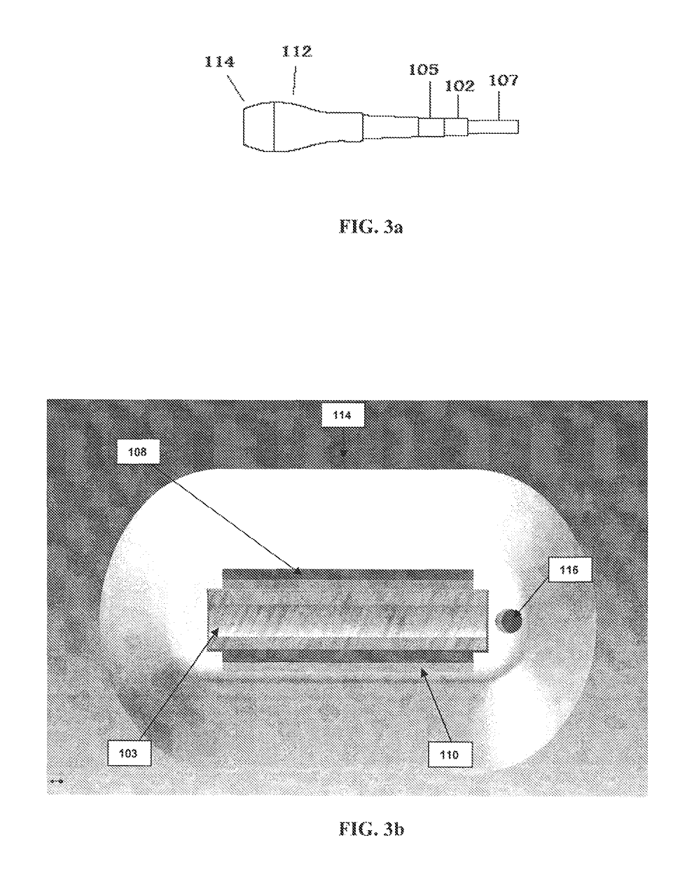

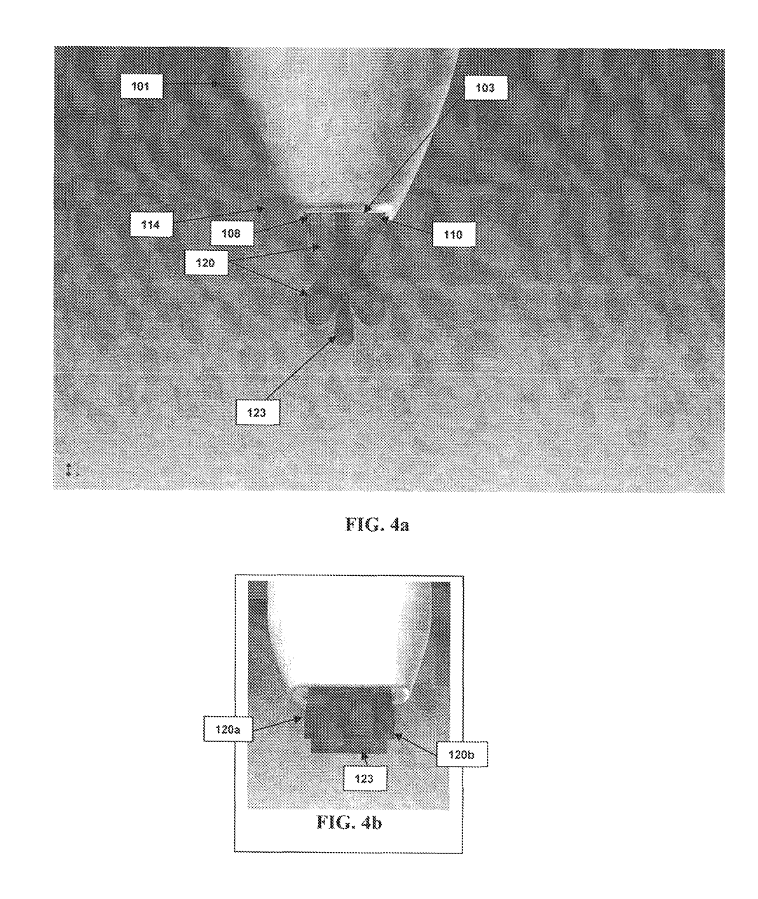

FIGS. 4a, 4b, and 5 show the interactions between the acoustic field or scan plane generated by the arrayed ultrasound transducer and the optical fields generated by the optical fibers of the scan head. In particular, the arrayed transducer 103 generates an acoustic field 123 that is perpendicular to the front surface 127 of the transducer 103. The bundles of optical fibers 104 and 106 that have been potted or otherwise integrated into the nosepiece 114 to form bars 108 and 110 that emit beams of light 120 onto the target. The optical fibers and resulting light beams can be placed at different angles relative to the illuminated tissue. The angle can be increased to the point that the light beams delivered to the subject are parallel with each other and also with the ultrasound beam. Typically, the bars of light 108 and 110 formed by the fiber optic bundles 104 and 106 are at an angle relative to the front surface 127 of the arrayed transducer 103 such that the light beams 120 emitted by the bundles intersect with each other and with the acoustic field 123 generated by the arrayed transducer 103. In certain embodiments, the light beams of the integrated photoacoustic transducer illuminate a volume of tissue which coincides with a rectangular region of the acoustic imaging plane of the arrayed transducer. As depicted in FIG. 5, the light beams 120 intersect the acoustic field 123 at region 125 of acoustic elevation focus, thereby allowing photoacoustic imaging over this region. Additionally, since light scatters strongly within tissue, photoacoustic imaging can be performed outside of the intersection region 125 as well, but resolution and sensitivity may be less optimal than within the intersection region 125.

As previously discussed, the epoxy or other resin material used to integrate the optical fibers into the nosepiece of the scan head may also be formed into lenses to focus the light beams produced by the fiber bundles. In particular, if the mold used to shape the epoxy or resin incorporates integral lens profiles, different molds can be tailored so that the potting epoxy or other resin results in lenses for each of the light bars that are used to focus the laser light from the optical fibers to an optimal position and to control divergence, intensity, and angle of incidence of the optical beams. Thus by changing the mold profile, different illumination patterns can be created using the same fiber bundles and acoustic transducer. Furthermore, if the potting process is done in a mold so that the resulting faces of the optical fibers are flush with the acoustic lens of the arrayed ultrasonic transducer, the resulting composite transducer will be easy to clean, and can be placed in as close proximity to the subject as possible.

FIG. 5 shows an embodiment of the scan head in which the epoxy or other resin material in front of the light emitting ends 108 and 110 of fiber bundles 104 and 106 is formed into lenses 128 and 130 that are flush with the acoustic lens 133, and refract and/or focus the laser light beams 120 emitted from the scan head into an optimal configuration with respect to the ultrasonic imaging plane. For example, the lenses 128 and 130 can be configured to provide for light beams 120 having a depth of focus that matches that of the acoustic field 123 generated by the arrayed ultrasound transducer 103. By using an optically transparent resin that has an index of refraction that is well matched to the index of refraction of the optical fibers, little loss of light occurs when the beam passes through the resin material in front of the optical fiber formed by the potting process. Additionally, the epoxy or resin used to form the lenses can also be used to fix the optical fibers at different elevation angles relative to the front surface of the transducer, thereby allowing a wider range of depths that the light beams can be focused at. This material also serves to protect the optical fibers against damage during use.

The ultrasound transducer used in the scan head is typically an arrayed transducer or another form of fixed transducer. “Fixed” transducers acquire ultrasound lines in a given scan plan without the need for the transducer to be physically moved along the scan plane. More specifically, the term “fixed” means that the transducer array does not utilize movement in its azimuthal direction during transmission or receipt of ultrasound in order to achieve its desired operating parameters, or to acquire a frame of ultrasound data. Moreover, if the transducer is located in a scan head or other imaging probe, the term “fixed” may also mean that the transducer is not moved in an azimuthal or longitudinal direction relative to the scan head, probe, or portions thereof during operation. A “fixed” transducer can be moved between the acquisitions of ultrasound frames, for example, the transducer can be moved between scan planes after acquiring a frame of ultrasound data, but such movement is not required for their operation. One skilled in the art will appreciate, however, that a “fixed” transducer can be moved relative to the object imaged while still remaining fixed as to the operating parameters. For example, the transducer can be moved relative to the subject during operation to change position of the scan plane or to obtain different views of the subject or its underlying anatomy.

Examples of arrayed transducers include, but are not limited to, a linear array transducer, a phased array transducer, a two-dimensional (2-D) array transducer, or a curved array transducer. A linear array is typically flat, i.e., all of the elements lie in the same (flat) plane. A curved linear array is typically configured such that the elements lie in a curved plane.

The transducer typically contains one or more piezoelectric elements, or an array of piezoelectric elements which can be electronically steered using variable pulsing and delay mechanisms. Suitable ultrasound systems and transducers that can be used with photoacoustic system of the invention include, but are not limited to those systems described in U.S. Pat. No. 7,230,368 (Lukacs et al.), which issued on Jun. 12, 2007; U.S. Patent Application Publication No.: US 2005/0272183 (Lukacs, et al.), which published Dec. 8, 2005; U.S. Patent Application Publication No. 2004/0122319 (Mehi, et al.), which published on Jun. 24, 2004; U.S. Patent Application Publication No. 2007/0205698 (Chaggares, et al.), which published on Sep. 6, 2007; U.S. Patent Application Publication No. 2007/0205697 (Chaggares, et al.), which published on Sep. 6, 2007; U.S. Patent Application no. 2007/0239001 (Mehi, et al.), which published on Oct. 11, 2007; U.S. Patent Application Publication No. 2004/0236219 (Liu, et al.), which published on Nov. 25, 2004; each of which is fully incorporated herein by reference.

A scan head of the invention may include a handle or otherwise be adapted for hand held use, or may be mounted onto to rail system, motor, or similar positioning device. The scan head cable is typically flexible to allow for easy movement and positioning of the transducer.

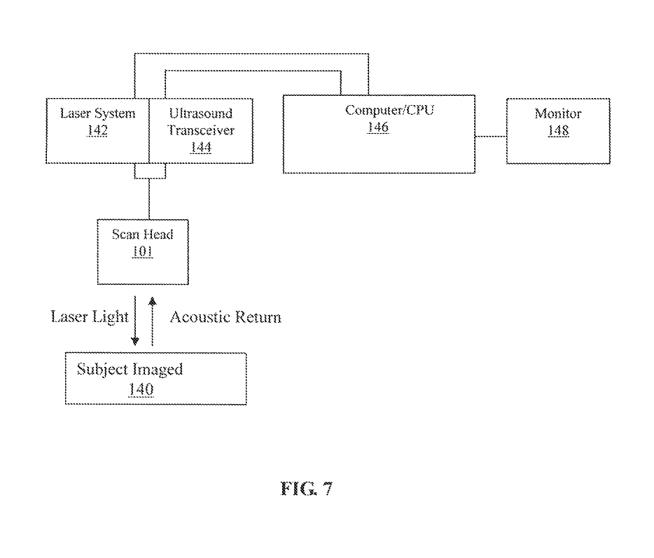

The scan head of the invention can be incorporated into a photoacoustic imaging system, such as that shown in FIG. 7, to provide for the creation of photoacoustic images of a subject. For example, the optical fibers of the scan head 101 can be connected to a laser system 142, such as a Rainbow NIR Integrated Tunable Laser System from OPOTEK (California, U.S.A.), that generates non-ionizing laser pulses. The laser-generating system in combination with the optical fibers in the scan head 101 directs laser pulses onto a subject 140, which results in the absorption of electromagnetic radiation thereby generating ultrasonic energy in the tissues and/or organs of the subject 140. The laser generating system may also contain a module for monitoring laser energy; both at the source of the laser output, and/or from returned light from the photoacoustic scan head through optical fibers. The transducer in the scan head 101 is connected via wires to a ultrasound transceiver or beamformer 144, and detects the ultrasonic waves generated by the laser light and sends this data to a central processing unit (e.g. computer) 146 that uses software to create two-dimensional and three-dimensional images of regions of interest within the subject, which are displayed on a monitor 148.

The integration of the optical fiber laser into the ultrasound transducer allows for both ultrasound imaging and photoacoustic imaging using the same device. When obtaining the photoacoustic images the ultrasound transducer is used primarily as a detector, but the transducer can be used to both send and receive ultrasound if the user wishes to operate the device in a purely ultrasound mode. Thus the system can, in some implementations, function as both a photoacoustic imaging system as well as an ultrasound imaging system.

The photoacoustic images can be formed by multiple pulse-acquisition events. Regions within a desired imaging area are scanned using a series of individual pulse-acquisition events, referred to as “A-scans” or ultrasound “lines.” Each pulse-acquisition event requires a minimum amount of time for the pulse of electromagnetic energy transmitted from the optical fibers to generate ultrasonic waves in the subject which then travel to the transducer. The image is created by covering the desired image area with a sufficient number of A-scan lines to provide a sufficient detail of the subject anatomy can be displayed. The number of and order in which the lines are acquired can be controlled by the ultrasound system, which also converts the raw data acquired into an image. Using a combination of hardware electronics and software instructions in a process known as “beamforming,” individual A-scans can be grouped together to form image data. Through a process of “scan conversion,” or image construction, the beamformed photoacoustic image data obtained is rendered so that a user viewing the display can view the subject imaged.

In one implementation of the invention, the ultrasound signals are acquired using receive beamforming methods such that the received signals are dynamically focused along an ultrasound line. The optical fibers are arranged such that each ultrasound line within the scan plane receives the same level of laser pulse intensity. A series of successive ultrasound lines are acquired to form a frame. For example, 256 ultrasound lines may be acquired, with the sequence of events for each line being the transmission of a laser pulse followed by the acquisition of ultrasound signals.

Line based image reconstruction methods are described in U.S. Pat. No. 7,052,460 issued May 30, 2006 and entitled “System for Producing an Ultrasound Image Using Line Based Image Reconstruction,” and in U.S. Patent Application Publication No. 2004/0236219 (Liu, et al.), which published on Nov. 25, 2004, each of which is incorporated fully herein by reference and made a part hereof. Such line based imaging methods can be incorporated to produce an image when a high frame acquisition rate is desirable, for example when imaging a rapidly beating mouse heart.

In another implementation of the invention, the ultrasound signals are acquired in an even faster manner with fewer laser pulses by acquiring A-scans on individual arrayed transducer elements simultaneously and then performing beamforming retrospectively, typically in software. Due to the homogeneous distribution of light from the light-emitting bars over the active area of the photoacoustic scan head, only a single laser pulse is required for illuminating the area of the image plane. Thus, rather than sending a laser pulse for each image line, a single laser pulse can be used to excite the tissue, and the returned ultrasound waves can be acquired on individual elements of the arrayed transducer. Depending on the number of available channels on the ultrasound system, more than one laser pulse may be required to cover the entire active area of the arrayed transducer. For example, in one embodiment of the invention, the ultrasound system contains 64 channels that are multiplexed to 256 ultrasound array elements. In this case, four laser pulses are used to collect A-scans on all 256 active elements. Through retrospective beam forming, however, image lines can be formed by taking groups of A-scans, known also as “apertures,” that exceed the limit of 64 channels on the system. Up to 256 elements could be used to form an aperture that would be beamformed into a single line, before repeating the process for the next image line. In practice, most lasers have very low pulse repetition rates (10-20 Hz), so using this process of retrospective beamforming is highly advantageous for improving photoacoustic imaging frame rates.

For 3D image acquisition, a motor may be used to move the ultrasound transducer with integrated fiber optic bundle in a linear motion to collect a series of frames separated by a predefined step size. The motor's motion range and step size may be set and/or adjusted by the user. Typically the step size is from about 10 μm to about 250 μm.

The motor typically moves the ultrasound transducer along a plane that runs perpendicular to the scan plane. These 2D images are then stacked and visualized as a volume using the standard 3D visualization tools. Methods for 3D photoacoustic image acquisition are described in more detail in U.S. Ser. No. 61/174,571, filed May 1, 2009, which is incorporated herein by this reference.

In addition to the scan head with ultrasound transducer and integrated fiber optic laser, the photoacoustic systems according to the invention typically include one or more of the following components: a processing system operatively linked to the other components that may be comprised of one or more of signal and image processing capabilities; a digital beamformer (receive and/or transmit) subsystems; analog front end electronics; a digital beamformer controller subsystem; a high voltage subsystem; a computer module; a power supply module; a user interface; software to run the beamformer and/or laser; software to process received data into two-and/or three-dimensional images; a scan converter; a monitor or display device; and other system features as described herein.

The block diagram in FIG. 7 shows a typical arrangement of components for the photoacoustic imaging system according to the invention. The system includes a scan head 101 which contains an arrayed transducer and integrated optical fibers for directing laser light generated by the laser system 142 onto the subject 140 to be imaged. An ultrasound transceiver/beamformer 144 is connected to elements of the active aperture of the arrayed transducer in the scan head 101, and is used to determine the aperture of the arrayed transducer.

During transmission, laser light emitted from the optical fibers of the scan head 101 penetrates into the subject 140 and generates ultrasound signals from within the tissues of the subject 140. The ultrasound signals are received by the elements of the active aperture of the arrayed transducer in the scan head 101 and converted into an analog electrical signal emanating from each element of the active aperture. The electrical signal is sampled to convert it from an analog to a digital signal in the ultrasound transceiver/beamformer 144. In some embodiments, the arrayed transducer in the scan head also has a receive aperture that is determined by a beamformer control, which tells a receive beamformer which elements of the array to include in the active aperture and what delay profile to use. The receive beamformer can be implemented using at least one field programmable gate array (FPGA) device. The photoacoustic imaging system can also comprise a transmit beamformer, which may also be implemented using at least one FPGA device. In yet another embodiment, the received photoacoustic signals on the elements of the array can be generated with fewer laser pulses by retrospectively beamforming the signal in software.

A central processing unit, e.g. a computer 146, has control software that runs the components of the system, including the laser system 142. The computer 146 also has software for processing received data, for example, using three-dimensional visualization software 108, to generate images based on the received ultrasound signals. The images are then displayed on a monitor 148 to be viewed by the user.

The components of the computer 146 can include, but are not limited to, one or more processors or processing units, a system memory, and a system bus that couples various system components including the beamformer 144 to the system memory. A variety of possible types of bus structures may be used, including a memory bus or memory controller, a peripheral bus, an accelerated graphics port, and a processor or local bus using any of a variety of bus architectures. By way of example, such architectures can include an Industry Standard Architecture (ISA) bus, a Micro Channel Architecture (MCA) bus, an Enhanced ISA (EISA) bus, a Video Electronics Standards Association (VESA) local bus, and a Peripheral Component Interconnects (PCI) bus also known as a Mezzanine bus. This bus, and all buses specified in this description can also be implemented over a wired or wireless network connection. This system can also be implemented over a wired or wireless network connection and each of the subsystems, including the processor, a mass storage device, an operating system, application software, data, a network adapter, system memory, an Input/Output Interface, a display adapter, a display device, and a human machine interface, can be contained within one or more remote computing devices at physically separate locations, connected through buses of this form, in effect implementing a fully distributed system.

The computer 146 typically includes a variety of computer readable media. Such media can be any available media that is accessible by the computer 146 and includes both volatile and non-volatile media, removable and non-removable media. The system memory includes computer readable media in the form of volatile memory, such as random access memory (RAM), and/or non-volatile memory, such as read only memory (ROM). The system memory typically contains data such as data and/or program modules such as operating system and application software that are immediately accessible to and/or are presently operated on by the processing unit.

The computer 146 may also include other removable/non-removable, volatile/non-volatile computer storage media. By way of example, a mass storage device which can provide non-volatile storage of computer code, computer readable instructions, data structures, program modules, and other data for the computer 146. For example, a mass storage device can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules can be stored on the mass storage device, including by way of example, an operating system and application software. Data including 2D and/or 3D images can also be stored on the mass storage device. Data can be stored in any of one or more databases known in the art. Examples of such databases include, DB2™, Microsoft™ Access, Microsoft™ SQL Server, Oracle™, mySQL, PostgreSQL, and the like. The databases can be centralized or distributed across multiple systems.

A user can enter commands and information into the computer 146 via an input device. Examples of such input devices include, but are not limited to, a keyboard, pointing device (e.g., a “mouse”), a microphone, a joystick, a serial port, a scanner, and the like. These and other input devices can be connected to the processing unit via a human machine interface that is coupled to the system bus, but may be connected by other interface and bus structures, such as a parallel port, game port, or a universal serial bus (USB). In an exemplary system of an embodiment according to the present invention, the user interface can be chosen from one or more of the input devices listed above. Optionally, the user interface can also include various control devices such as toggle switches, sliders, variable resistors and other user interface devices known in the art. The user interface can be connected to the processing unit. It can also be connected to other functional blocks of the exemplary system described herein in conjunction with or without connection with the processing unit connections described herein.

A display device or monitor 148 can also be connected to the system bus via an interface, such as a display adapter. For example, a display device can be a monitor or an LCD (Liquid Crystal Display). In addition to the display device 148, other output peripheral devices can include components such as speakers and a printer which can be connected to the computer 146 via Input/Output Interface.

The computer 146 can operate in a networked environment using logical connections to one or more remote computing devices. By way of example, a remote computing device can be a personal computer, portable computer, a server, a router, a network computer, a peer device or other common network node, and so on. Logical connections between the computer 146 and a remote computing device can be made via a local area network (LAN) and a general wide area network (WAN). Such network connections can be through a network adapter. A network adapter can be implemented in both wired and wireless environments. Such networking environments are commonplace in offices, enterprise-wide computer networks, intranets, and the Internet. The remote computer may be a server, a router, a peer device or other common network node, and typically includes all or many of the elements already described for the computer 146. In a networked environment, program modules and data may be stored on the remote computer. The logical connections include a LAN and a WAN. Other connection methods may be used, and networks may include such things as the “world wide web” or Internet.

Aspects of the exemplary systems shown in the Figures and described herein can be implemented in various forms including hardware, software, and a combination thereof. The hardware implementation can include any or a combination of the following technologies, which are all well known in the art: discrete electronic components, a discrete logic circuit(s) having logic gates for implementing logic functions upon data signals, an application specific integrated circuit having appropriate logic gates, a programmable gate array(s) (PGA), field programmable gate array(s) (FPGA), etc. The software comprises an ordered listing of executable instructions for implementing logical functions, and can be embodied in any computer-readable medium for use by or in connection with an instruction execution system, apparatus, or device, such as a computer-based system, processor-containing system, or other system that can fetch the instructions from the instruction execution system, apparatus, or device and execute the instructions.

The photoacoustic imaging systems and methods of the invention can be used in a wide variety of clinical and research applications to image various tissues, organs, (e.g., heart, kidney, brain, liver, blood, etc.) and/or disease conditions of a subject. For example, the described embodiments enable in vivo visualization, assessment, and measurement of anatomical structures and hemodynamic function in longitudinal imaging studies of small animals. The systems can provide images having very high resolution, image uniformity, depth of field, adjustable transmit focal depths, multiple transmit focal zones for multiple uses. For example, the photoacoustic image can be of a subject or an anatomical portion thereof, such as a heart or a heart valve. The image can also be of blood and can be used for applications including evaluation of the vascularization of tumors. The systems can be used to guide needle injections.

For imaging of small animals, it may be desirable for the transducer to be attached to a fixture during imaging. This allows the operator to acquire images free of the vibrations and shaking that usually result from “free hand” imaging. The fixture can have various features, such as freedom of motion in three dimensions, rotational freedom, a quick release mechanism, etc. The fixture can be part of a “rail system” apparatus, and can integrate with the heated mouse platform. A small animal subject may also be positioned on a heated platform with access to anesthetic equipment, and a means to position the transducer relative to the subject in a-flexible manner.

The systems can be used with platforms and apparatus used in imaging small animals including “rail guide” type platforms with maneuverable probe holder apparatuses. For example, the described systems can be used with multi-rail imaging systems, and with small animal mount assemblies as described in U.S. patent application Ser. No. 10/683,168, entitled “Integrated Multi-Rail Imaging System,” U.S. patent application Ser. No. 10/053,748, entitled “Integrated Multi-Rail Imaging System,” U.S. patent application Ser. No. 10/683,870, now U.S. Pat. No. 6,851,392, issued Feb. 8, 2005, entitled “Small Animal Mount Assembly,” and U.S. patent application Ser. No. 11/053,653, entitled “Small Animal Mount Assembly,” each of which is fully incorporated herein by reference.

Small animals can be anesthetized during imaging and vital physiological parameters such as heart rate and temperature can be monitored. Thus, an embodiment of the system may include means for acquiring ECG and temperature signals for processing and display. An embodiment of the system may also display physiological waveforms such as an ECG, respiration or blood pressure waveform

The described embodiments can also be used for human clinical, medical, manufacturing (e.g., ultrasonic inspections, etc.) or other applications where producing a three-dimensional photoacoustic image is desired.

As used in this description and in the following claims, “a” or “an” means “at least one” or “one or more” unless otherwise indicated. In addition, the singular forms “a”, “an”, and “the” include plural referents unless the content clearly dictates otherwise. Thus, for example, reference to a composition containing “a compound” includes a mixture of two or more compounds.

As used in this specification and the appended claims, the term “or” is generally employed in its sense including “and/or” unless the content clearly dictates otherwise.

The recitation herein of numerical ranges by endpoints includes all numbers subsumed within that range (e.g. 1 to 5 includes 1, 1.5, 2, 2.75, 3, 3.80, 4, and 5).

Unless otherwise indicated, all numbers expressing quantities of ingredients, measurement of properties and so forth used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings of the present invention. At the very least, and not as an attempt to limit the scope of the claims, each numerical parameter should at least be construed in light of the number of reported significant digits and by applying ordinary rounding techniques. Any numerical value, however, inherently contains certain errors necessarily resulting from the standard deviations found in their respective testing measurements.

Various modifications and alterations to the invention will become apparent to those skilled in the art without departing from the scope and spirit of this invention. It should be understood that the invention is not intended to be unduly limited by the specific embodiments and examples set forth herein, and that such embodiments and examples are presented merely to illustrate the invention, with the scope of the invention intended to be limited only by the claims attached hereto.

The complete disclosures of the patents, patent documents, and publications cited herein are hereby incorporated by reference in their entirety as if each were individually incorporated.

What is claimed is:

1. A photoacoustic scan head comprising:
(i) an arrayed ultrasound transducer having a front surface for detecting ultrasound waves from a target,
(ii) a housing comprising a nosepiece for containing the arrayed ultrasound transducer, and
(iii) a plurality of optical fibers for directing laser light to the target, wherein light-emitting portions of the fibers are positioned adjacent to the front surface of the arrayed ultrasound transducer and integrated into the nosepiece of the housing with an optically transparent resin.

2. The photoacoustic scan head of claim 1, wherein the ultrasound transducer is a linear array transducer.

3. The photoacoustic scan head of claim 1, wherein at least a portion of the optical fibers is joined together to form a bundle.

4. The photoacoustic scan head of claim 3, wherein at least a portion of the optical fibers is bundled together with one or more electrical wires that run to the arrayed ultrasound transducer.

5. The photoacoustic scan head of claim 3, wherein the optical fibers in the nosepiece of the housing are arranged into at least two bundles each of which has a light-emitting end positioned to deliver a beam of light to the target.

6. The photoacoustic scan head of claim 5, wherein the light-emitting ends of the two bundles of optical fibers are positioned on either side of the arrayed ultrasound transducer.

7. The photoacoustic scan head of claim 5, wherein the light-emitting end of each bundle of optical fibers is in the form of a rectangular bar of fibers.

8. The photoacoustic scan head of claim 5, wherein the light emitting end of each bundle of optical fibers is in the form of a circle.

9. The photoacoustic scan head of claim 6, wherein the light-emitting end of each bundle of optical fibers is positioned at an angle relative to the front surface of the arrayed ultrasound transducer so that the beam of light generated by each bundle of optical fibers intersects a plane that runs perpendicular to the front face of the transducer.

10. The photoacoustic scan head of any of claim 1, further comprising a real-time capable photo-sensor for monitoring pulse-to-pulse laser energy.

11. The photoacoustic scan head of claim 10, wherein the photo-sensor monitors pulse-to-pulse backscatter intensity.

12. The photoacoustic scan head of claim 10, wherein the photo-sensor is integrated into the nosepiece of the housing using the same optically transparent resin used to integrate the optical fibers into the housing.

13. The photoacoustic scan head of claim 10, further comprising a plurality of photo-sensors distributed around the transducers for monitoring pulse-to-pulse energy variation at different region of the arrayed ultrasound transducer.

14. The photoacoustic scan head of claim 10, further comprising a separate group of optical fibers that is positioned next to the photo-sensor and emits a beam of light onto an area of the target adjacent to an acoustic field generated by the ultrasound transducer.

15. The photoacoustic scan head of claim 5, wherein the light-emitting ends of the two bundles of optical fibers are positioned on either side of the arrayed ultrasound transducer, and are capable of guiding backscattered light back to a photo-sensor located outside the housing of the scan head for monitoring of pulse-to-pulse energy.

16. The photoacoustic scan head of claim 5, further comprising additional optical fibers dedicated solely to directing light back to the photosensor, wherein the additional optical fibers are positioned either within the existing optical fiber bundles or around the exterior of the existing optical fiber bundles, and are capable of guiding backscattered light back to a photo-sensor located outside the housing of the scan head for monitoring of pulse-to-pulse energy.

17. The photoacoustic scan head of claim 1, wherein the optically transparent resin is polymer resin.

18. The photoacoustic scan head of claim 15, wherein the translucent resin is an epoxy resin.

19. The photoacoustic scan head of claim 1, wherein the index of refraction of the resin matches the index of refraction of the optical fibers.

20. The photoacoustic scan head of claim 1, wherein the ultrasound transducer is integrated into the housing using the same transparent resin used to integrate the optical fibers into the housing.

21. The photoacoustic scan head of claim 5, wherein the translucent resin acts as a lens to focus the beams of light emitted by the optical fibers.

22. The photoacoustic scan head of claim 21, wherein the beams of light have a depth of focus that matches that of the acoustic field generated by the arrayed ultrasound transducer.

23. The photoacoustic scan head of claim 1, wherein the ultrasound transducer receives and transmits ultrasound at a frequency from about 15 MHz to about 100 Mhz.

24. The photoacoustic scan head of claim 1, wherein the ultrasound transducer receives and transmits ultrasound at a frequency of at least 20 MHz.

25. A photoacoustic imaging system comprising:
(i) a scan head of claim 1,
(ii) a laser system to generating pulses of non-ionizing light, wherein the laser system is connected to the optical fibers of the scan head,
(iii) an ultrasonic transceiver connected to the transducer of the scan head,
(iv) a computer for controlling system components and processing received ultrasound data into an image, and
(v) a monitor for displaying the image.
